(19) United States
(12) Patent Application Publication
(10) Pub. No.: US 2026/0335562 A1
(43) Pub. Date: Oct. 29, 2026

(54) AUXILIARY DEVICE FOR AN INJECTION DEVICE

(71) Applicant: SANOFI, Paris (FR)

(72) Inventors: Michael HELMER, Frankfurt am Main (DE); Michael SCHABBACH, Frankfurt am Main (DE)

(21) Appl. No.: 19/469,233

(22) PCT Filed: Mar. 22, 2024
(86) PCT No.: PCT/EP2024/057757

§ 371 Date: Sep. 25, 2025

(30) Foreign Application Priority Data
Mar. 28, 2023 (EP) 23315063.0

(51) Int. Cl. A61M 5/315

(52) U.S. Cl. CPC A61M 5/31568; A61M 2205/3306; A61M 2205/3561; A61M 2205/60; A61M 2205/8212

(57) ABSTRACT

An auxiliary device for an injection device is disclosed. The auxiliary device includes a body having a fastening structure configured to detachably couple the auxiliary device to the injection device and a handle portion configured for user gripping. An electronic circuit disposed in the body includes a processor and a first transceiver and is selectively switchable between an operating mode and an inactive mode to reduce power consumption when not in use. A first actuating element is provided on the handle portion and is operably connected to the electronic circuit. Actuation of the first actuating element by a user activates the electronic circuit, for example to enable wireless communication, control, or data exchange via the transceiver. The detachable fastening and handle configuration facilitate ergonomic use, while the mode switching and handle-mounted actuator provide intuitive activation and efficient operation.

### CROSS-REFERENCE TO RELATED APPLICATIONS

The present application is the national stage entry of International Patent Application No. PCT/EP2024/057757, filed on Mar. 22, 2024, and claims priority to European Application No. EP23315063.0, filed on Mar. 28, 2023, the disclosures of which are incorporated herein by reference.

### TECHNICAL FIELD

The present disclosure relates to auxiliary devices for use with injection devices, such as syringes, safety syringes or pen-type injectors. in a further aspect the present disclosure relates to an injection system comprising an injection device and an auxiliary device. In a further aspect the disclosure relates to a method of monitoring use or preparation of an injection device or injection system for injecting a medicament.

### BACKGROUND

Drug delivery devices for setting and dispensing a single or multiple doses of a liquid medicament are as such well-known in the art. Generally, such devices have substantially a similar purpose as that of an ordinary syringe.

Drug delivery devices, such as pen-type injectors, have to meet a number of user-specific requirements. For instance, with patients suffering chronic diseases, such as diabetes, the patient may be physically infirm and may also have impaired vision. Suitable drug delivery devices especially intended for home medication therefore need to be robust in construction and should be easy to use. Furthermore, manipulation and general handling of the device and its components should be intelligible and easy understandable. Such injection devices should provide setting and subsequent dispensing of a dose of a medicament of equal or variable size. Moreover, a dose setting as well as a dose dispensing procedure must be easy to operate and has to be unambiguous.

A patient suffering from a particular disease may require a certain amount of a medicament to either be injected via a pen-type injection syringe.

Some drug delivery or injection devices provide selecting of a dose of a medicament of variable size and injecting a dose previously set. Other injection devices provide setting and dispensing of a fixed dose. Here, the amount of medicament that should be injected in accordance to a given prescription schedule is always the same and does not change or cannot be changed over time.

Some injection devices are implemented as reusable injection devices offering a user to replace a medicament container, such as a cartridge. Other injection devices are implemented as a disposable injection device. With disposable injection devices it is intended to discard the entirety of the injection device when the content, i.e. the medicament, has been used up.

In order to control and to supervise administering of medication conducted by users or patients themselves it is desirable to provide an automated detecting and logging of a repeated and regular use of the drug delivery device. A rather automated recording of doses injected by a user would offer a significant advantage over a manual dose logging in terms of security and convenience.

There exist numerous add-on devices or auxiliary device that are generally configured for use with injection devices and which offer an electronic detection and monitoring of single or repeated dose injection procedures.

Typically, such add-on devices or auxiliary devices can be detachably connected to an injection device. An add-on device typically comprises a sensor, detector or detector arrangement operable to detect a date and/or time when the user sets or injects a dose of the medicament. Some add-on devices also provide a quantitative measurement of a size of a dose currently set or dispensed. Some add-on devices are intended for use with a series of injection devices. This may particularly apply with disposable injection devices, that are intended to become discarded after use or after the medicament located therein has been used up.

There may arise situations, in which a user may be unaware of a correct us of the injection device or auxiliary device. Moreover, there may arise situations, in which a user inadvertently activates an auxiliary device without having an intention to do so. Such inadvertent activation or use of the auxiliary device may lead to a wrong or inconsistent recording, logging or monitoring of an injection procedure.

It is therefore desirable to provide an improved auxiliary device for use with an injection device, by way of which patient safety can be enhanced and which offers improved functionality of its use and handling. It is a further aim to provide an auxiliary device, which serves to assist a user to conduct an injection procedure and/or which gives feedback to a user with regards to an actual or preceding injection procedure. Furthermore, the auxiliary device should provide enhanced communication capabilities with external electronic devices, such as smart watches, smart phones, tablet computers or the like.

### SUMMARY

In one aspect there is provided an auxiliary device for an injection device. The auxiliary device comprises a body. The body comprises a fastening structure or an attachment structure for detachably fastening to the injection device. The body further comprises a handle portion for gripping by a user. The auxiliary device further comprises an electronic circuit. The electronic circuit comprises a processor and a first transceiver. The electronic circuit is switchable between an operating mode and an inactive mode or sleep mode. The auxiliary device further comprises a first actuating element, which is provided on the handle portion. The first actuating element is operably connected to the electronic circuit. It is operable to activate the electronic circuit when actuated by the user.

By arranging the first actuating element on the handle portion and by the operability of the first actuating element to activate the electronic circuit it may serve as a kind of on/off switch for the auxiliary device. By integrating and/or by providing the first actuating element on the handle portion a user intending to make use of the auxiliary device when fixed to the injection device may automatically switch on the auxiliary device when gripping the handle portion of the auxiliary device.

This way, the auxiliary device can be rather intuitively operated, i.e. switched on and/or switched off, e.g. concurrently with the gripping of the handle portion by the user. With some examples the processor is implemented as a microprocessor or as a micro controller. It may be operably connected with the first actuating element. Insofar and by operating, i.e. by actuating the first actuating member by a user the processor of the electronic circuit can be selectively activated. By releasing the first actuating element the electronic circuit may be automatically deactivated or transferred into a sleep mode, in which the processor or at least a portion thereof is inactive. When deactivated or when in the sleep mode, energy consumption of the processor is substantially reduced compared to situations, in which the processor and/or the electronic circuit is in the operating mode.

In some examples and when the electronic circuit is in the operating mode the electronic circuit, e.g. its processor and/or or its first transceiver, is operable to record, to log or to monitoring a dose injecting operation of the injection device. Here, the electronic circuit may be operable to monitor a date and/or a time of an injection procedure. In some examples the electronic circuit may be operable to measure a size of a dose injection. In some examples the electronic circuit may be operable to record a date and/or a time of an injection procedure together with a size of a dose currently set and/or injected.

With some examples the injection device is implemented as a syringe, e.g. as a prefilled syringe and/or as a safety syringe. A safety syringe typically comprises a prefilled syringe arranged or assembled in a safety mechanism. The safety mechanism typically provides a longitudinal displacement of an injection needle of the syringe relative to a housing component or relative to a shield of the injection device after completion of an injection procedure. This way the distal injecting end of the injection needle can be protected e.g. by a housing portion, by a shield or by a needle guard. This way a likelihood of stitch damages upon use of the injection device can be substantially reduced.

According to a further example the electronic circuit is in the inactive mode as long as the first actuating element is released or remains unoperated. Accordingly, the electronic circuit can be activated only by actuating the actuating element. In this way, and by having the first actuating element arranged on or inside the handle portion of the body of the auxiliary device, an activation of the electronic circuit requires a respective gripping of the handle portion, thereby actuating the actuating element. In this way, an operation of the auxiliary device always requires that a user correctly uses the handle portion and grips or holds the auxiliary device with the injection device attached thereto in an intended way, thereby automatically actuating the actuating element.

According to a further example the electronic circuit of the auxiliary device comprises a power source. When released the first actuating element is operable to disconnect the processor from the power source. Here, the first actuating element may be implemented or may serve as a switch by way of which the processor can be selectively connected or disconnected to and from the power source.

When the actuating element is released the power source can be disconnected from the processor. Vice versa, the processor can be connected to the power source and can be operated or provided with sufficient electrical energy by actuating the first actuating element. Otherwise, and as long as the first actuating element is or remains released the processor may be disconnected from the power source. The power source may be typically implemented as an electrical battery, which may be a rechargeable battery or exchangeable battery.

According to a further example the auxiliary device comprises a second actuating element provided on the handle portion. Also, the second actuating element is operably connected to the electronic circuit and is operable to activate the electronic circuit when actuated by the user. Typically, the electronic circuit is in and remains in the inactive mode as long as the second actuating element is released or remains unoperated by a user. Also, the second actuating element may be operable to disconnect the processor from the power source. Insofar, the first and the second actuating element may be implemented rather identically or at least in a likewise manner.

By providing a first and a second actuating element on the handle portion the user may be prompted to actuate the first and the second actuating elements simultaneously in order to activate the electronic circuit. Otherwise, and if only one of the first and second actuating elements may be actuated by the user the electronic circuit will remain in the inactive mode. Switching the electronic circuit from the inactive mode into the active mode or operating mode requires an actuation of both, the first actuating element and the second actuating element, typically in an at least temporally overlapping time interval.

By providing a first and a second actuating element on the handle portion the user of the auxiliary device is prompted to actuate both, namely the first actuating element as well as the second actuating element simultaneously or at least in temporally overlapping time intervals. Otherwise, the electronic circuit may remain in the inactive mode. This way, the user of the auxiliary device intending to activate the auxiliary device is encouraged or prompted to actuate, e.g. to depress the first and the second actuating element substantially simultaneously or to actuate the two actuating element during an overlapping time interval. This way, an inadvertent use or inadvertent operation of the auxiliary device can be effectively suppressed. With only one actuating element there may be still a certain risk that the user not intending to make use of the auxiliary device by accident actuates the first or the second actuating element. With the requirement of a simultaneous or a temporally overlapping actuation of the first actuating element and of the second actuating element an inadvertent operation of the auxiliary device can be effectively prevented.

Insofar and by providing a first actuating element and a second actuating element an inadvertent switching of the electronic circuit from an inactive mode into the operating mode can be effectively suppressed. At least, a likelihood of an inadvertent activation of the electronic circuit switching from the inactive mode into the operating mode can be substantially reduced.

According to a further example the electronic circuit is switchable from the inactive mode into the operating mode or active mode only by actuating the first actuating element and the second actuating element. Actuation of the first actuating element and of the second actuating element has to be conducted simultaneously or at least within a predefined overlapping time interval. Otherwise, and e.g. by a sequential or temporarily non-overlapping actuation of the first actuating element and the second actuating element the electronic circuit may remain in the inactive mode.

According to a further example the electronic circuit remains in the operating mode as long as the first actuating element and the second actuating element are both actuated by the user. Releasing only one of the first actuating element and the second actuating element may switch the electronic circuit from the operating mode into the inactive mode.

Accordingly, and with a user intending to monitor an injection process by making use of the auxiliary device connected to an injection device the user is obliged to keep the first actuating element and the second actuating element actuated, e.g. depressed, during the entire dose injection process. A premature release of only one of the first actuating element and/or the second actuating element may switch the electronic circuit into the inactive mode. Hence, recording or monitoring of a medicament dose injection process may be corrupted or may be indicated to be improper.

Generally, switching of the electronic circuit into the inactive mode may include at least one of a power disconnection of the processor or switching of the processor into a sleep mode or idle mode, in which modes the processor consumes less energy compared to when it is in the operating mode.

According to a further example the electronic circuit is switchable from the operating mode or active mode into the inactive mode by releasing one or both of the first actuating element and the second actuating element.

Typically, the first and the second actuating elements are arranged on the handle portion in such a way, that an intended use of the auxiliary device or injection device the user is in constant contact with the first actuating element and with the second actuating element. Typically, the user releases the at least one or both of the first actuating element and the second actuating element after having completed a dose injection procedure. Here, the electronic circuit with the first actuating element and with the optional second actuating element may provide an automatic deactivation of the electronic circuit and hence a rather automated switch-off of the electronic circuit into the inactive mode when at least one or both of the first actuating element is no longer actuated. In this way, electrical energy and hence electrical power as provided by the power source of the auxiliary device can be saved. In this way the lifetime of the power source of the auxiliary device, e.g. of a battery, could be the prolonged.

According to a further example the first actuating element comprises a first switch. Also the second actuating element may comprise a second switch. The switches may be manually actuatable by a user, e.g. in the course of gripping the handle portion of the body of the auxiliary device. Implementation of respective switches is rather straight forward and simple. A mechanical or electromechanical switch may also provide a haptic feedback to a user when actuated.

According to a further example at least one of the first and the second switches may be electrically connected with the processor and with the power source. One terminal of such a switch may be connected to the processor. Another terminal of the respective switch may be connected to the power source. Insofar and by closing of the respective switch the processor may be provided with electrical power and may wake up to provide the intended operation.

According to a further example the first switch and the second switch are connected in series. A serial arrangement of a first switch and a second switch provides a kind of a logic AND gate. Here, the electrically conductive connection between the power source and the processor may be only provided by closing the first switch and the second switch.

Opening or releasing at least one of the first and the second switches may effectively disconnect the processor from the power source. The electronic circuit will then be switched into the inactive mode.

A logic and gate may be likewise implemented with the electronic circuit. Hence, the electronic circuit may comprise a respective AND gate operable by the first and second actuating elements. Such an AND gate may be even integrated into the processor. Respective terminals of the AND gate may be selectively actuatable by the user of the auxiliary device or injection device.

According to a further example the handle portion of the body of the auxiliary device comprises a distally facing gripping surface. The first actuating element is arranged on the distally facing gripping surface. In the present context a distal direction extends or points towards the injection end of the injection device and hence towards the biological tissue to be pierced by an injection needle of the injection device. Accordingly, a proximal direction faces opposite the distal direction.

With some examples the handle portion provides the distally facing gripping surface, which may be e.g. in mechanical contact with a middle finger and an index finger of a hand of a user intending to inject a dose of the medicament by making use of the injection device attached to the auxiliary device. Typically, and when appropriately attached or mutually fastened, the injection device is fixed to the auxiliary device at least with regards to a longitudinal direction, e.g. extending along a distance between the distal end and the proximal end of the injection device.

By arranging the first actuating element on the distally facing gripping surface of the handle portion it may be automatically actuated by a user when provide or exerting a counterforce onto the auxiliary device in the course of urging a stopper of a barrel of the injection device towards the distal direction for injecting the medicament. In this way and by providing the first actuating element on the distally facing gripping surface it may be automatically actuated by a user when the user intends to use the auxiliary device in connection with the injection device to set and/or to inject a dose of the medicament.

With a further example the handle portion comprises a gripping flange protruding from the fastening structure or body of the auxiliary device. The first actuating element and the second actuating element are typically provided on the distally facing gripping surface, which extends on opposite sides of the fastening structure or body of the auxiliary device. Here, the first actuating element may be provided on a first portion of the gripping flange extending outwardly along a first radial direction from the fastening structure or the body. The second actuating element may be provided on a second portion of the gripping flange extending diametrically opposite to the first portion of the gripping flange. It may extend along a second radial direction opposite the first radial direction.

By having first and second actuating elements of a common distally facing gripping surface but on diametrically opposite radially outwardly extending flange portions of a gripping flange the first and second actuating element may be intuitively actuated by a user when using the gripping flange to hold the auxiliary device for and/or during an injection procedure.

With another example of the auxiliary device the body of the auxiliary device comprises a receptacle to receive at least a portion of the injection device. The receptacle of the body provides a rather easy and intuitive mutual fastening of the auxiliary device and the injection device. The receptacle may be part of the fastening structure or may constitute the fastening structure.

With a further example the body of the auxiliary device comprises a through opening sized to receive at least a portion of the injection device there through. The receptacle and/or the through opening of the body of the auxiliary device may comprise at least one longitudinal abutment, by way of which the injection device, when received in the receptacle and/or in the through opening, can be fixed relative to the body at least with regards to the longitudinal direction. Here, and when the injection device is for instance implemented as a syringe or as a safety syringe a flange portion protruding radially outwardly from the respective syringe or syringe body may longitudinally abut or engage with a respective stop face as provided on or in the receptacle or through opening of the body of the auxiliary device.

According to a further example the fastening structure of the body comprises a mechanical coding to mate with a complementary shaped mechanical counter coding of the injection device. With injection devices of elongated and rather radially symmetric shape, such as syringes or the like injection devices comprising a tubular-shaped barrel, syringe body or housing, the receptacle or through opening as provided on or in the body of the auxiliary device may be generally operable to receive the injection device therein in an arbitrary orientation with regards to the longitudinal axis of the injection device as an axis of rotation. Now, and with a mechanical coding, e.g. at an edge or on the inside of the receptacle or through opening to mate with a complementary-shaped mechanical counter coding, e.g. provided on an outside facing surface of the injection device there can be provided an unequivocal and hence well-defined mutual fastening or arrangement and attachment of the injection device to the fastening structure and hence to the body of the auxiliary device.

In this way, the injection device may be mounted or attached to the auxiliary device in only one or in a limited number of well-defined mutual orientations, thereby improving patient safety as well as a proper handling of the injection device in connection with the auxiliary device.

According to a further example the auxiliary device comprises a first sensor operable to detect at least one of a position and a movement of a plunger or stopper of the injection device relative to one of a barrel, a housing and a shield or needle guard of the injection device when the auxiliary device is fastened to the injection device, or when the injection device is fastened to the auxiliary device. By way of the first sensor, a configuration or operation of the injection device can be automatically detected. It can be detected, monitored or quantitatively measured.

Typically, the first sensor is part of the electronic circuit. The first sensor may be operably connected to the processor. Sensor signals as provided by the sensor are typically processable by the processor in order to detect a configuration or an operational status of the injection device.

With another example the auxiliary device also comprises a second sensor operable to detect at least one of a position and a movement of a plunger or stopper of the injection device relative to one of a barrel, a housing, a shield and a needle guard of the injection device when the auxiliary device is fastened to the injection device. By way of a second sensor, a further configuration or a further operational status of the injection device can be detected, monitored or quantitatively measured.

With some examples the first sensor may be operable to detect and/or to quantitatively measure a first configuration or a first operational status of the injection device, e.g. prior to inject a dose of the medicament. The second sensor may be particularly operable to detect a second configuration or to detect a second operational status of the injection device, e.g. after completion of a dose injection procedure.

Insofar and with a first sensor and with a second sensor at least two different configurations and/or two different operations of one and the same injection device can be precisely detected. This way, it may be possible to detect the beginning of an injection procedure, e.g. by way of the first sensor and to detect termination or completion of a dose injection procedure by the second sensor.

Providing a first sensor and a second sensor also provides a redundancy of the functionality of the auxiliary device. Even if one of the first sensor and the second sensor should fail, the other sensor may still provide respective sensor data being indicative of the configuration or operational status of the injection device.

However, with the first sensor and with the second sensor different dedicated configurations or operational states of the injection device can be precisely detected or monitored.

With another example the first sensor and the second sensor are located offset with regard to a longitudinal direction of the injection device. Hence, the first sensor may be implemented as a proximal sensor and the second sensor may be implemented as a distal sensor, or vice versa. The first sensor may be the indicative of a proximal position of the plunger or stopper relative to the barrel, the housing and/or relative to a shield of the injection device. Likewise, the second sensor may be operable to generate sensor signals being indicative of a distal position of the plunger or stopper of the injection device relative to one of the barrel, the housing and the shield or needle guard of the injection device.

The first and second sensors located longitudinally offset with respect to each other are of particular use for injection devices intended for a single use, such as prefilled syringes or prefilled safety syringes. Here, the plunger or stopper of the injection device is subject to a single, typically to a single distally directed movement relative to a barrel, a housing and/or a shield of the injection device. Hence, after completion of an injection the configuration of the injection device distinguishes from the configuration of the injection device before setting or injecting of a dose.

With a further example the auxiliary device comprises a third sensor operable to detect at least one of a presence, a position and a movement of a protective cap of the injection device when the auxiliary device is fastened or attached to the injection device. With some examples the injection device may be equipped with a protective cap, e.g. covering or enclosing the injection needle. Prior to injection it is typically required to remove the protective cap in order to expose the distal end of the injection needle. Typically, the third sensor is operably connected to the processor and is operable to generate and to transmit respective sensor signals to the processor being indicative of the presence, hence of the position and/or of the movement of the protective cap.

By way of the third sensor, it can be detected if the protective cap is initially present and/or if the protective cap has been correctly detached from the injection device before conducting of an injection procedure. Respective sensor signals as provided by at least one of the first sensor, the second sensor and the third sensor are typically processed by the processor of the auxiliary device. By way of a respective signal processing, it can be determined if a user correctly uses the injection device.

With some examples the electronic circuit comprises a detection unit, which is operably connected with the processor and which is further connected to our comprises at least one of the first sensor, the second sensor and the third sensor. The detection unit may provide a pre-processing of signals generated by the individual sensors. The detector unit may further control operation of the various sensors.

With some examples the electronic circuit further comprises an actuation unit, which is operably connected with the first and second actuating elements. The actuation unit may comprise the first and the second actuating elements. It may be provided in form of an integrated circuit and/or in form of an electromechanically implemented switching device According to a further example the first sensor and the second sensor are of equal sensor type. Hence, the first and the second sensors may operate on the same sensor principle. The third sensor may be implemented in a different way. Hence, the type of the third sensor may distinguish from the type of the first and the second sensors. With some examples, the first sensor, the second sensor and the third sensor are of equal sensor type. With some examples, at least one of the sensors is implemented as one of an electrical contact sensor, as a capacitive sensor, as a magnetic sensor or as an optical sensor. With some examples at least one of the first sensor, the second sensor and the third sensor is of a contactless sensor type. A contactless sensor may be of particular benefit since it does not interfere with the mechanical operation of the injection device or auxiliary device.

With some examples the first sensor comprises an optical sensor. It may comprise a light-sensitive detector, such as a photodiode. Such optical sensors can be easily integrated into an electronic circuit in a rather cost-efficient way. They allow a rather compact design of the electronic circuit and can be operated at a comparatively low power level.

With another example the second sensor also comprises an optical sensor. The second sensor may be implemented identical to the first optical sensor.

With some examples also the third sensor may be implemented as an optical sensor.

The optical sensors may be implemented as a light switch. The optical sensors may be implemented to constantly detect a light intensity reflected from a component of the injection device. As soon as the respective component of the injection device, such as the plunger, the stopper, the barrel, the housing or the shield should be subject to a displacement the optical sensor may record a respective amendment in the transmitted or reflected light from the moving component and may thus detect a respective position variation or movement of the device component.

According to a further example the first sensor comprises a first light source.

Here, the light detector of the first sensor may be sensitive for the wavelength of spectrum of the first light source. The first light source may be directed towards or onto a detectable component of the injection device, such as the plunger, the stopper, the barrel, the housing, the shield or the needle guard of the injection device.

With a further example the first light source is aligned with a first dedicated portion of the injection device, i.e. with a dedicated portion of a selected or dedicated component of the injection device when the injection device is correctly or duly attached to the body.

The first light source is operable to illuminate the dedicated portion of the injection device. When for instance the first light source operates in the visible spectral range the correct alignment or arrangement of the injection device with the auxiliary device may lead to a respective alignment of the first light source with the dedicated portion or component of the injection device, which may be then illuminated with radiation generated by the first light source. Such an illumination of a dedicated portion or component of the injection device may assist a user in using the injection device. With some examples the dedicated portion or component of the injection device may be translucent or transparent. Then, an illumination as provided by the first light source may be immediately apparent to a user of the auxiliary device and the injection device.

According to a further example of the auxiliary device the second sensor comprises a second light source. The second light source may be operable to transmit or emit electromagnetic radiation of a second wavelength, wherein the second wavelength distinguishes or differs from a first wavelength of electromagnetic radiation generated, emitted or transmitted by the first light source. Typically, the light detector of the second optical sensor is susceptible to the spectrum or wavelength of the radiation generated by the second light source.

When the wavelength of the first light source and the wavelength of the second light source distinguish and when the sensitivity of the light detectors of the first and second optical sensors varies accordingly it can be provided that the first optical sensor operates at a first wavelength or first spectrum and that the second optical sensor operates at a second wavelength or second spectrum of electromagnetic radiation. The first and second sensors may be then insensitive to radiation provided or emitted by the light source of the other one of the first and second optical sensors.

With first and second wavelength substantially distinguishing from each other there can be avoided any crosstalk between the first optical sensor and the second optical sensor

With another example the second light source may be aligned with a second dedicated portion or component of the injection device when the injection device is correctly attached to the body of the auxiliary device. The second light source is operable to illuminate the dedicated portion or dedicated component of the injection device in the same or in a like manner as described above in connection with the first light source. With the first and the second light sources operating in the visible spectral range they may provide different colors, that can be distinguished by a user of the auxiliary device.

Hence, the optical sensors with their light sources could not only be used to detect one of a position and a movement of a dedicated portion or component of the injection device but can be likewise used to illuminate the dedicated or selected portions or components of the injection device in the same or in different colors to thereby instruct or to assist a user in using the auxiliary device and/or the injection device.

According to a further example at least one of the first light source and the second light source is operable to generate visible light of variable color. Typically, the optical sensors are operably connected to the processor of the electronic circuit. This way operation of the optical sensors with their respective light sources can be controlled by the processor. With a light source of variable color the processor may be further operable to select a wavelength and/or to activate the respective light source to emit electromagnetic radiation of a selected or selectable color.

Use of different colors, e.g. red, green, blue or yellow may be of particular benefit to illuminate dedicated portions or component of the injection device to thereby assist a user in using the auxiliary device. For instance, a dedicated portion or component of the injection device illuminated in green may indicate to a user that the auxiliary device and/or the injection device is ready to use. A respective illumination of the same or of another dedicated portion or component of the injection device in a different color, e.g. in blue or red may indicate to a user, that e.g. the auxiliary device or the injection device cannot be or should not be used again.

With a further example the first sensor of the auxiliary device is sensitive to a first wavelength of electromagnetic radiation and the second center is sensitive to a second wavelength of electromagnetic radiation. The first wavelength and the second wavelengths are different and/or comprise at least a non-overlapping spectral range. In this way, the first optical sensor with the first light source may operate at a first wavelength and the second optical sensor with the second light source may operate at the second wavelengths, which measurably distinguishes from the first wavelength.

According to a further example the electronic circuit comprises a clock operable to provide a clock signal being indicative of a date and/or a time. The electronic circuit further comprises a memory operable to store electronic data. The memory as well as the clock are operably connected to the processor. The processor is further operable to process a first sensor signal from the first sensor, which first sensor signal is indicative of a first configuration or operation state of the injection device. Furthermore, the processor is operable to store the first sensor signal and the clock signal in form of electronic data in the memory.

Typically, the processor is operable to combine the first sensor signal and the clock signal concurrent with the first sensor signal and to store the combination of the clock signal and the first sensor signal in the memory. In this way, the first sensor signal can be provided with a timestamp and respective data stored in the memory is indicative of a point of time, at which the first sensor signal has been generated or processed.

With a further example the processor is operable to process a second sensor signal from the second sensor. The second sensor signal is indicative of a second configuration or operation state of the injection device. Furthermore, the processor is operable to store the second sensor signal and the clock signal in form of electronic and/or digital data in the memory. Insofar, the processor is operable to combine a clock signal or a timestamp with sensor signals obtainable from the first sensor and the second sensor, respectively.

The first configuration or operation state may be indicative of the start of an injection procedure. The second configuration or operation state of the injection device as detectable by the second sensor may be indicative of a termination or completion of a respective injection procedure.

This way, and by storing respective sensor signals concurrently with clock signals in the memory there can be provided a dosing history being indicative of the beginning and the termination of an injection procedure.

With further examples it is also conceivable, that generation or processing of one of the first sensor signal and the second sensor signal triggers recording or storing of a respective clock signal. This may particularly apply for such auxiliary devices and injection devices, wherein the injection device can be used only once and wherein the injection device is operable to inject a dose of a fixed size. Here, repeated use of the injection device or of a number of injection devices of equal type may only require to record a point in time, at which an injection procedure has successfully completed.

With a further example the processor is operable to compare a first clock signal with a second clock signal. The first clock signal is indicative of the generation of the first sensor signal. The second clock signal is indicative of a generation of the second sensor. Here, the processor is operable to generate at least one of an alert signal and an indication signal if a time interval between generation of the first clock signal and the second clock signal is larger than a predefined time interval. In this way, the electronic circuit and hence the processor is operable to verify, if first and second sensor signal will be generated within a predefined time interval.

With an intended and correct use of the injection device, respective first and second sensor signals may be generated or provided e.g. at the beginning and at the completion of a dose injection procedure. If the time interval between the generation or recording of first and second sensor signal and hence of respective first and second clock signal is larger than the predefined time interval this may be an indication, that the injection procedure has either not terminated successfully or that the injection procedure was not correctly executed or conducted.

An alert signal or indication signal may be further processed to generate at least one of a visual, an audible and/or haptic alert. Alert generation may be provided by the electronic circuit of the auxiliary device, which may be this equipped with respective alert generating hardware, such as one of a visual indicator, a speaker or a vibration unit.

With some examples such an alert signal may be transmitted to an external electronic device, such as a smartwatch, a smart phone or a tablet computer, e.g. via the first transceiver, which external electronic device is provided with respective hardware to generate a respective audible, visual or haptic alert or a respective indication signal.

With a further example the first transceiver is operable to transmit or to exchange data with an external electronic device. This way, the auxiliary device may be void of any signaling devices or hardware but may be simply operable to communicate with the external electronic device to make use of respective input and output devices or with respective input and output hardware of the external electronic device in order to communicate with a user of the auxiliary device or injection device.

The first transceiver may be implemented as a wireless transceiver. It may be configured to transmit and/or to receive radiofrequency signals. In particular, it may be operable to communicate with the external electronic device according to an established communication standard, such as Bluetooth, Bluetooth low energy (BLW) or a Wi-Fi standard.

According to a further example the auxiliary device comprises a second transceiver operable to read an electronic identifier provided on or inside the injection device. The second transceiver may also belong to the electronic circuit. The first transceiver as well as the second transceiver may be operably connected with the processor. The first transceiver and/or the second transceiver may be controllable by the processor of the electronic circuit of the auxiliary device. The second transceiver is particularly operable to read an electronic information, hence an electronic identifier provided on or inside the injection device.

The second transceiver may be implemented as a wireless transceiver. It may wirelessly connect with the electronic identifier of the injection device. The electronic identifier may comprise information about a medicament. The information stored in the electronic identifier may comprise at least one of a name of the medicament, a name of the pharmaceutical substance of the medicament, a concentration of the medicament, a batch number, a LOT number, a manufacturing date, a manufacturing site or manufacturing location, an expiry date and/or a temperature, at which the medicament should be stored or has been stored.

The electronic identifier as provided on or inside the injection device may comprise an electronic tag. The electronic identifier may comprise one of an RFID tag and an NFC tag. The second transceiver of the auxiliary device may comprise a respective reader complementary or correspondingly implemented to the electronic identifier as provided on or inside the injection device.

With a further example the second transceiver comprises a near field transceiver. While the first transceiver comprises a local range transceiver. A transmission range of the first transceiver may be larger than a transmission range of the second transceiver. Moreover, a correct reading of the electronic identifier of the injection device may require a correct mutual assembly, mounting or fastening of the auxiliary device and the injection device. For this, the mechanical coding and the complementary shaped mechanical counter coding as provided on or in the fastening structure and the injection device may be configured such, that a successful mutual fastening and attachment of the auxiliary device and the injection device comes along with a correct and intended orientation of the electronic identifier of the injection device with the second transceiver of the auxiliary device.

With the auxiliary device comprising a first transceiver and a second transceiver the auxiliary device may operate or may provide a relay station for a wireless transmission between the electronic identifier of the injection device with an external electronic device of a user intending to make use of the auxiliary device and the injection device. The electronic circuit with the first transceiver and the second transceiver may be operable to read at least part of the information stored in the electronic identifier of the injection device and to transmit respective information to the external electronic device via the first transceiver. Since the first transceiver comprises a transmission range exceeding the transmission range of the second transceiver the auxiliary device is operable to provide a kind of a range extender for the wireless data transmission between the electronic identifier and the external electronic device.

Accordingly and with a further example the electronic circuit is operable to read identification data of the electronic identifier of the injection device via the second transceiver and to transmit the identification data to the external electronic device via the first transceiver.

According to a further example the auxiliary device comprises an indicator to align with a visual identifier provided on or inside the injection device when the injection device is correctly fastened or assembled to the body of the auxiliary device. The indicator corresponds or interacts with the visual identifier and allows to visually indicate information to a user via the visual identifier.

With some examples the visual identifier as provided on or inside the injection device is or comprises a light guide or a light guiding structure, which can be activated or illuminated by the indicator of the auxiliary device. Here, the indicator may comprise a light source, which is operably connected to the processor and which is operable by the processor.

When the injection device and the auxiliary device are correctly assembled and/or mutually attached the light source of the indicator may at least partially overlap with the visual identifier and may illuminated the visual identifier, which illumination may be directly visible on a portion of the injection device non-obstructed by the auxiliary device.

With some examples the visual identifier of the injection device may comprise at least one longitudinal stripe extending in longitudinal direction. One end of the longitudinal stride may overlap with the light source or the indicator or light source of the auxiliary device, which when activated may induce a visible light signal propagating through and eliminating the visual identifier of the injection device. The light source of the indicator may be a single color or multicolor light source, such as a light emitting diode (LED).

In another aspect the present disclosure also relates to an injection system. The injection system comprises an injection device. The injection device comprises a barrel filled with a medicament and being sealed towards a proximal direction by a stopper. The stopper is movable in a distal direction relative to the barrel in order to expel the medicament through an outlet of the barrel. Typically, the outlet of the barrel is in fluid communication with an injection needle. With some examples the injection needle is connected to the outlet of the barrel or constitutes or contributes to the outlet of the barrel. The outlet is typically provided at the distal end of the barrel.

The injection system further comprises an auxiliary device as described above.

With some examples the injection device and the auxiliary device are mutually fixed and assembled. With other examples the injection system is provided as a kit, wherein the injection device and the auxiliary device are provided separately in an unconnected or disassembled state. Here, a user or a health care professional may assemble or attach the auxiliary device to the injection device; or vice versa.

Since the injection system comprises an auxiliary device as described above, all features, effects and benefits as described above in connection with the auxiliary device equally apply to the injection system; and vice versa.

According to a further example of the injection system the injection device is one of a prefilled syringe, a prefilled safety syringe. With further examples the injection device is a prefilled injection pen. With further examples the injection device is a reusable device. With further examples the injection device is a disposable device, which upon use is to be discarded in its entirety.

In another aspect the present disclosure relates to a method of monitoring use of preparation of an injection device or injection system for injecting a medicament. The method comprises the step of providing of an injection device operable to inject the medicament and an auxiliary device as described above. Furthermore, the method comprises the step of activating the electronic circuit of the auxiliary device by actuating the first actuating element. With some examples the method is applicable with an auxiliary device as described above featuring or comprising a second actuating element operably connected to the electronic circuit and operable to activate the electronic circuit. Activating the electronic circuit may then comprise actuating the first actuating element during a first time interval and actuating the second actuating element during a second time interval. Here, the first time interval and the second time interval at least partially overlap in the temporal domain.

In other words, activating the electronic circuit includes a rather simultaneous activation of the first actuating element and the second actuating element or at least actuating the first and the second actuating elements over or during an overlapping time interval.

With another example the method further includes a deactivation of the electronic circuit, hence a switching of the electronic circuit into an inactive mode. Here, at least one of the first actuating element and the second actuating element is released and the electronic circuit is consequently switched into the inactive mode, in which the auxiliary device does not consume electric power or consumes a reduced amount of electric power compared to its operating mode.

In another independent aspect and e.g. according to a second embodiment, the present disclosure relates to an auxiliary device for an injection device. The auxiliary device comprises a body. The body comprises a fastening structure for detachably fastening to the injection device. The auxiliary device comprises an electronic circuit comprising a processor.

The electronic circuit further comprises a first sensor and a second sensor. The first sensor and the second center are operably connected to the processor of the electronic circuit. The first sensor is operable to detect at least one of a position and a movement of a plunger or stopper of the injection device relative to one of a barrel, a housing and a shield or needle guard of the injection device when the auxiliary device is fastened to the injection device.

With a further example the second sensor is operable to detect at least one of a position and a movement of a plunger or stopper of the injection device relative to one of a barrel, a housing and a shield or needle guard of the injection device when the auxiliary device is fastened to the injection device.

Typically, the first sensor is operable to detect a first configuration or a first operational state of the injection device when fastened to the auxiliary device.

Correspondingly, the second sensor is operable to detect a second configuration or a second operational state of the injection device when suitably fastened to the auxiliary device.

Apart from that, the first sensor and the second sensor may be implemented in a way as described above with the auxiliary device and the injection system The first sensor and the second sensor may comprise an optical sensor or, e.g. equipped with a dedicated first or second light source.

Moreover, the optical sensors may be aligned with a first or second dedicated portion or component of the injection device. The respective light sources of at least one of the first and the second sensors may be operable to illuminate the respective dedicated portion or component of the injection device. The first and second sensors of the auxiliary device may be implemented in a way as described above in greater detail. Here and contrary to the auxiliary device as described above, the second embodiment of the auxiliary device may be void of a first actuating element and/or of an optional second actuating element. In other words and with the second embodiment of the auxiliary device, the first and second actuating elements as described above may be only optional.

Generally, the scope of the present disclosure is defined by the content of the claims. The injection system, the auxiliary device and/or the injection device and respective methods as described herein are not limited to specific embodiments or examples but comprises any combination of elements of different embodiments or examples. Insofar, the present disclosure covers any combination of claims and any technically feasible combination of the features disclosed in connection with different examples or embodiments.

In the present context the term ‘distal’ or ‘distal end’ relates to an end of the injection device that faces towards an injection site of a person or of an animal. The term ‘proximal’ or ‘proximal end’ relates to an opposite end of the injection device, which is furthest away from an injection site of a person or of an animal.

The terms “drug” or “medicament” are used synonymously herein and describe a pharmaceutical formulation containing one or more active pharmaceutical ingredients or pharmaceutically acceptable salts or solvates thereof, and optionally a pharmaceutically acceptable carrier. An active pharmaceutical ingredient (“API”), in the broadest terms, is a chemical structure that has a biological effect on humans or animals. In pharmacology, a drug or medicament is used in the treatment, cure, prevention, or diagnosis of disease or used to otherwise enhance physical or mental well-being. A drug or medicament may be used for a limited duration, or on a regular basis for chronic disorders.

As described below, a drug or medicament can include at least one API, or combinations thereof, in various types of formulations, for the treatment of one or more diseases. Examples of API may include small molecules having a molecular weight of 500 Da or less; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA, small interfering RNA (siRNA), ribozymes, genes, and oligonucleotides. Nucleic acids may be incorporated into molecular delivery systems such as vectors, plasmids, or liposomes. Mixtures of one or more drugs are also contemplated.

The drug or medicament may be contained in a primary package or “drug container” adapted for use with a drug delivery device. The drug container may be, e.g., a cartridge, syringe, reservoir, or other solid or flexible vessel configured to provide a suitable chamber for storage (e.g., short-or long-term storage) of one or more drugs. For example, in some instances, the chamber may be designed to store a drug for at least one day (e.g., 1 to at least 30 days). In some instances, the chamber may be designed to store a drug for about 1 month to about 2 years. Storage may occur at room temperature (e.g., about 20 °C), or refrigerated temperatures (e.g., from about −4 °C to about 4 °C). In some instances, the drug container may be or may include a dual-chamber cartridge configured to store two or more components of the pharmaceutical formulation to-be-administered (e.g., an API and a diluent, or two different drugs) separately, one in each chamber. In such instances, the two chambers of the dual-chamber cartridge may be configured to allow mixing between the two or more components prior to and/or during dispensing into the human or animal body. For example, the two chambers may be configured such that they are in fluid communication with each other (e.g., by way of a conduit between the two chambers) and allow mixing of the two components when desired by a user prior to dispensing. Alternatively or in addition, the two chambers may be configured to allow mixing as the components are being dispensed into the human or animal body.

The drugs or medicaments contained in the drug delivery devices as described herein can be used for the treatment and/or prophylaxis of many different types of medical disorders. Examples of disorders include, e.g., diabetes mellitus or complications associated with diabetes mellitus such as diabetic retinopathy, thromboembolism disorders such as deep vein or pulmonary thromboembolism. Further examples of disorders are acute coronary syndrome (ACS), angina, myocardial infarction, cancer, macular degeneration, inflammation, hay fever, atherosclerosis and/or rheumatoid arthritis. Examples of APIs and drugs are those as described in handbooks such as Rote Liste 2014, for example, without limitation, main groups 12 (anti-diabetic drugs) or 86 (oncology drugs), and Merck Index, 15th edition.

Examples of APIs for the treatment and/or prophylaxis of type 1 or type 2 diabetes mellitus or complications associated with type 1 or type 2 diabetes mellitus include an insulin, e.g., human insulin, or a human insulin analogue or derivative, a glucagon-like peptide (GLP-1), GLP-1 analogues or GLP-1 receptor agonists, or an analogue or derivative thereof, a dipeptidyl peptidase-4 (DPP4) inhibitor, or a pharmaceutically acceptable salt or solvate thereof, or any mixture thereof. As used herein, the terms “analogue” and “derivative” refers to a polypeptide which has a molecular structure which formally can be derived from the structure of a naturally occurring peptide, for example that of human insulin, by deleting and/or exchanging at least one amino acid residue occurring in the naturally occurring peptide and/or by adding at least one amino acid residue. The added and/or exchanged amino acid residue can either be codable amino acid residues or other naturally occurring residues or purely synthetic amino acid residues. Insulin analogues are also referred to as “insulin receptor ligands”. In particular, the term “derivative” refers to a polypeptide which has a molecular structure which formally can be derived from the structure of a naturally occurring peptide, for example that of human insulin, in which one or more organic substituent (e.g. a fatty acid) is bound to one or more of the amino acids. Optionally, one or more amino acids occurring in the naturally occurring peptide may have been deleted and/or replaced by other amino acids, including non-codeable amino acids, or amino acids, including non-codeable, have been added to the naturally occurring peptide.

Examples of insulin analogues are Gly(A21), Arg(B31), Arg(B32) human insulin (insulin glargine); Lys(B3), Glu(B29) human insulin (insulin glulisine); Lys(B28), Pro(B29) human insulin (insulin lispro); Asp(B28) human insulin (insulin aspart); human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Examples of insulin derivatives are, for example, B29-N-myristoyl-des(B30) human insulin, Lys(B29) (N-tetradecanoyl)-des(B30) human insulin (insulin detemir, Levemir®); B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N-(N-palmitoyl-gamma-glutamyl)-des(B30) human insulin, B29-N-omega-carboxypentadecanoyl-gamma-L-glutamyl-des(B30) human insulin (insulin degludec, Tresiba®); B29-N-(N-lithocholyl-gamma-glutamyl)-des(B30) human insulin; B29-N-(w-PATENT carboxyheptadecanoyl)-des(B30) human insulin and B29-N-(w-carboxyheptadecanoyl) human insulin.

Examples of GLP-1, GLP-1 analogues and GLP-1 receptor agonists are, for example, Lixisenatide (Lyxumia®), Exenatide (Exendin-4, Byetta®, Bydureon®, a 39 amino acid peptide which is produced by the salivary glands of the Gila monster), Liraglutide (Victoza®), Semaglutide, Taspoglutide, Albiglutide (Syncria®), Dulaglutide (Trulicity®), rExendin-4, CJC-1134-PC, PB-1023, TTP-054, Langlenatide/HM-11260C (Efpeglenatide), HM-15211, CM-3, GLP-1 Eligen, ORMD-0901, NN-9423, NN-9709, NN-9924, NN-9926, NN-9927, Nodexen, Viador-GLP-1, CVX-096, ZYOG-1, ZYD-1, GSK-2374697, DA-3091, MAR-701, MAR709, ZP-2929, ZP-3022, ZP-DI-70, TT-401 (Pegapamodtide), BHM-034. MOD-6030, CAM-2036, DA-15864, ARI-2651, ARI-2255, Tirzepatide (LY3298176), Bamadutide (SAR425899), Exenatide-XTEN and Glucagon-Xten.

An example of an oligonucleotide is, for example: mipomersen sodium (Kynamro®), a cholesterol-reducing antisense therapeutic for the treatment of familial hypercholesterolemia or RG012 for the treatment of Alport syndrom. Examples of DPP4 inhibitors are Linagliptin, Vildagliptin, Sitagliptin, Denagliptin, Saxagliptin, Berberine.

Examples of hormones include hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, and Goserelin.

Examples of polysaccharides include a glucosaminoglycane, a hyaluronic acid, a heparin, a low molecular weight heparin or an ultra-low molecular weight heparin or a derivative thereof, or a sulphated polysaccharide, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium. An example of a hyaluronic acid derivative is Hylan G-F 20 (Synvisc®), a sodium hyaluronate.

The term “antibody”, as used herein, refers to an immunoglobulin molecule or an antigen-binding portion thereof. Examples of antigen-binding portions of immunoglobulin molecules include F(ab) and F(ab′)2 fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region. The term antibody also includes an antigen-binding molecule based on tetravalent bispecific tandem immunoglobulins (TBTI) and/or a dual variable region antibody-like binding protein having cross-over binding region orientation (CODV).

The terms “fragment” or “antibody fragment” refer to a polypeptide derived from an antibody polypeptide molecule (e.g., an antibody heavy and/or light chain polypeptide) that does not comprise a full-length antibody polypeptide, but that still comprises at least a portion of a full-length antibody polypeptide that is capable of binding to an antigen. Antibody fragments can comprise a cleaved portion of a full length antibody polypeptide, although the term is not limited to such cleaved fragments. Antibody fragments that are useful in the present invention include, for example, Fab fragments, F(ab′)2 fragments, scFv (single-chain Fv) fragments, linear antibodies, monospecific or multispecific antibody fragments such as bispecific, trispecific, tetraspecific and multispecific antibodies (e.g., diabodies, triabodies, tetrabodies), monovalent or multivalent antibody fragments such as bivalent, trivalent, tetravalent and multivalent antibodies, minibodies, chelating recombinant antibodies, tribodies or bibodies, intrabodies, nanobodies, small modular immunopharmaceuticals (SMIP), binding-domain immunoglobulin fusion proteins, camelized antibodies, and VHH containing antibodies. Additional examples of antigen-binding antibody fragments are known in the art.

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen.

Examples of antibodies are anti PCSK-9 mAb (e.g., Alirocumab), anti IL-6 mAb (e.g., Sarilumab), and anti IL-4 mAb (e.g., Dupilumab).

Pharmaceutically acceptable salts of any API described herein are also contemplated for use in a drug or medicament in a drug delivery device. Pharmaceutically acceptable salts are for example acid addition salts and basic salts.

Those of skill in the art will understand that modifications (additions and/or removals) of various components of the APIs, formulations, apparatuses, methods, systems and embodiments described herein may be made without departing from the full scope and spirit of the present invention, which encompass such modifications and any and all equivalents thereof.

An example drug delivery device may involve a needle-based injection system as described in Table 1 of section 5.2 of ISO 11608-1: 2014(E). As described in ISO 11608-1: 2014(E), needle-based injection systems may be broadly distinguished into multi-dose container systems and single-dose (with partial or full evacuation) container systems. The container may be a replaceable container or an integrated non-replaceable container.

As further described in ISO 11608-1: 2014(E), a multi-dose container system may involve a needle-based injection device with a replaceable container. In such a system, each container holds multiple doses, the size of which may be fixed or variable (pre-set by the user). Another multi-dose container system may involve a needle-based injection device with an integrated non-replaceable container. In such a system, each container holds multiple doses, the size of which may be fixed or variable (pre-set by the user).

As further described in ISO 11608-1: 2014(E), a single-dose container system may involve a needle-based injection device with a replaceable container. In one example for such a system, each container holds a single dose, whereby the entire deliverable volume is expelled (full evacuation). In a further example, each container holds a single dose, whereby a portion of the deliverable volume is expelled (partial evacuation). As also described in ISO 11608-1: 2014(E), a single-dose container system may involve a needle-based injection device with an integrated non-replaceable container. In one example for such a system, each container holds a single dose, whereby the entire deliverable volume is expelled (full evacuation). In a further example, each container holds a single dose, whereby a portion of the deliverable volume is expelled (partial evacuation).

### BRIEF DESCRIPTION OF THE DRAWINGS

In the following, examples of an injection system comprising an injection device and an auxiliary device for monitoring use of the injection device will be described in greater detail by making reference to the drawings, in which:

FIG. 1 shows an example of an injection system comprising an auxiliary device and an injection device,

FIG. 2 shows the example of FIG. 1 with the auxiliary device attached to the injection device and before conducting an injection procedure,

FIG. 3 is illustrative of the injection system of FIG. 2 after completion of a dose injection procedure,

FIG. 4 shows a configuration of the injection system according to FIG. 3 after retracting the needle into the housing of the injection device.

FIG. 5 schematically illustrates a longitudinal cross-section through the injection device according to the configuration of FIG. 2,

FIG. 6 is a longitudinal cross-section of the device of FIG. 5 that corresponds with the situation of FIG. 3 and

FIG. 7 shows a further longitudinal cross-section through the injection system after retraction of the needle into the housing of the injection device,

FIG. 8 shows a perspective illustration of a fastening structure of the auxiliary device,

FIG. 9 is a perspective view of the auxiliary device according to FIG. 8 as seen from below,

FIG. 10 schematically illustrates a use configuration of the injection system when operated by a user,

FIG. 11 schematically illustrated a data exchange between an external electronic device and the auxiliary device,

FIG. 12 schematically shows another example of an auxiliary device suitable for use with an injection device,

FIG. 13 is a block diagram of an example of the electronic circuit, and

FIG. 14 is a flowchart of a method of monitoring use of preparation of an injection device by making use of the auxiliary device.

### DETAILED DESCRIPTION

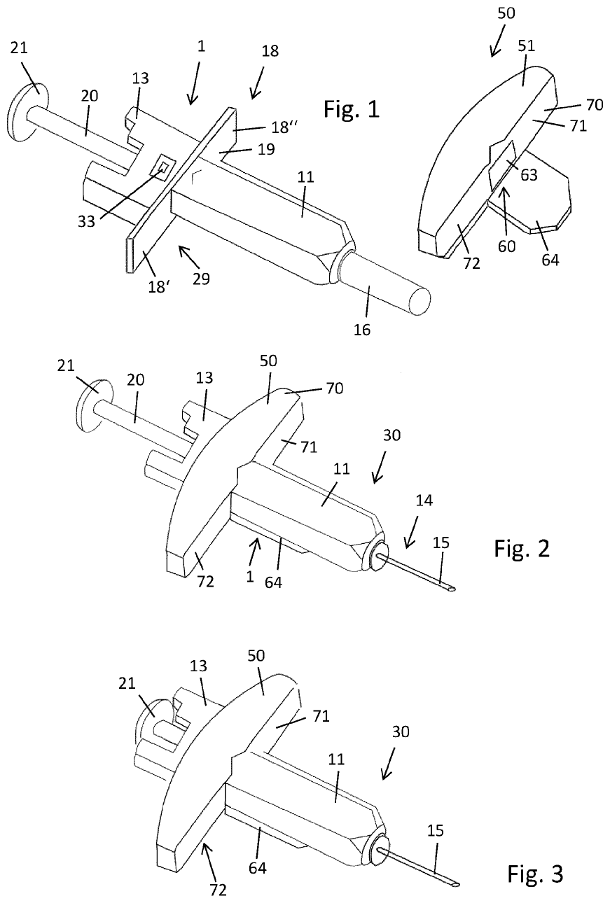

In the sequence of FIGS. 1-7 a scenario of using an injection device 1 with an auxiliary device 50 is schematically illustrated. With the illustrated example the injection device 1 comprises a kind of a syringe, e.g. implemented as a safety syringe. The injection device 1 comprises a barrel 10 filled with a liquid injectable medicament 8. Towards a proximal longitudinal direction 3 the barrel 10 is sealed by a stopper 9, which is movable in distal direction 2 relative to the barrel 10. The barrel 10 may be of tubular shape. It may comprise an outlet 14 at its distal end. The outlet 14 may be provided with an injection needle 15, fastened to the outlet 14 of the barrel 10. Towards the proximal end or proximal direction 3 the barrel 10 terminates with a radially outwardly extending or protruding flange portion 17, which when the injection device may be conventionally, could be used as a finger grip for an index finger and a middle finger of a user to suitably hold the syringe while the user may use a thumb 5 of the same hand 4 to press against a plunger 20 protruding proximally from the barrel 10.

The stopper 9 may be integrally formed with a longitudinally extending plunger 20. The plunger 20 may comprise an elongated rod extending in longitudinal direction (z). With some examples the stopper 9 and the plunger 20 may be provided as separate pieces. Here, the plunger 20 may be used to exert a distally directed pressure onto the stopper 9 in order to move the stopper in distal direction 2 relative to the barrel 10, thereby expelling a well-defined amount of the liquid medicament 8 through the outlet 14 and into the biological tissue when the injection needle 15 has penetrated or pierced the tissue. The stopper 9 may comprise an elastomeric material to hermetically seal the interior of the barrel 10.

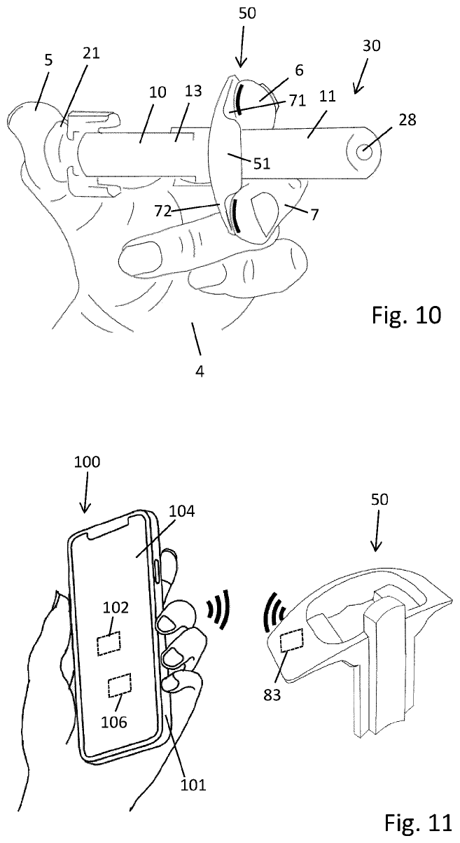

The proximal end of the plunger 20 is typically provided with a radially widened plunger flange 21. The plunger flange 21 is typically configured to be depressed by a thumb 5 of a user as e.g. illustrated in FIG. 10. With the example of FIGS. 1-7 the injection device 1 is implemented as a safety syringe. It is provided with a housing 11, which is of substantially elongated or tubular shape. The housing 11 is sized to accommodate the barrel 10 as well as a spring element 27. The spring element 27 is operably engaged with the housing 11 and with the barrel 10. It may be configured to induce a relative longitudinal motion between the barrel 10 and the housing 11, particularly with a completion of a dose injection procedure.

The barrel 10 may be secured against a longitudinal motion relative to the housing 11 by an interlock 24. In the example as illustrated in FIG. 6, the interlock 24 is provided at or near a proximal end of the barrel 10 or housing 11. The housing 11, which may be also of sleeve-like shape, comprises a counter latch element 26 to releasably engage with a latch element 25 as provided on the barrel 10. The latch element 25 and the counter latch element 26 are initially in an engaged configuration by way of which a longitudinal displacement of the barrel 10 relative to the housing 11 is impeded or locked.

It is only upon the plunger 10 reaching a distal end configuration, in which the plunger flange 21 engages the counter latch element 26 in such a way that the counter latch element 26 is deformed of pivoted so as to give way for the latch element 25. Once the latch elements 25 is released from the counter latch element 26, mechanical energy stored in the spring element 27 can be released thus leading to a longitudinal movement or longitudinal displacement of the barrel 10 relative to the housing 11.

The outlet 14 or the needle 15 initially protruding from the distal end of the housing 11 and reaching through a through opening 28 as provided on a distal end face of the housing 11 is or are retracted into the interior of the housing 11 as illustrated in FIG. 7. Here, the injection device 1′ is in a shielded or protected configuration, in which the injection device 1 cannot be no longer used and wherein the distally protruding tip of the injection needle 15 is protected by the housing 11.

As further illustrated in FIGS. 5 and 1, and in an initial configuration the outlet 14 and/or the injection needle 15 may be protected or covered by a separate protective cap 16.

Before conducting an injection procedure, the protective cap 16 should be detached and removed from the outlet 14 or needle 15. In the presently illustrated example the counter latch element 26 may be located at or near a radially outwardly extending flange portion 18 of the housing 11. The flange portion 18 may be complementary shaped to the flange portion 17. The proximal end of the flange portion 18 and/or the proximal end of the housing 11 may be further provided with a beveled edge 23, which is configured to engage with the distally facing side or edge of the plunger flange 21 as the plunger 20 reaches a distal end position as shown in FIG. 6. When the plunger flange 21 abuts against the beveled edge 23, the beveled edge 23 and hence the counter latch element 26 is urged radially outwardly, thereby disengaging from the latch elements 25, which may be simply provided by the radially outwardly extending flange portion 17 of the barrel 10.

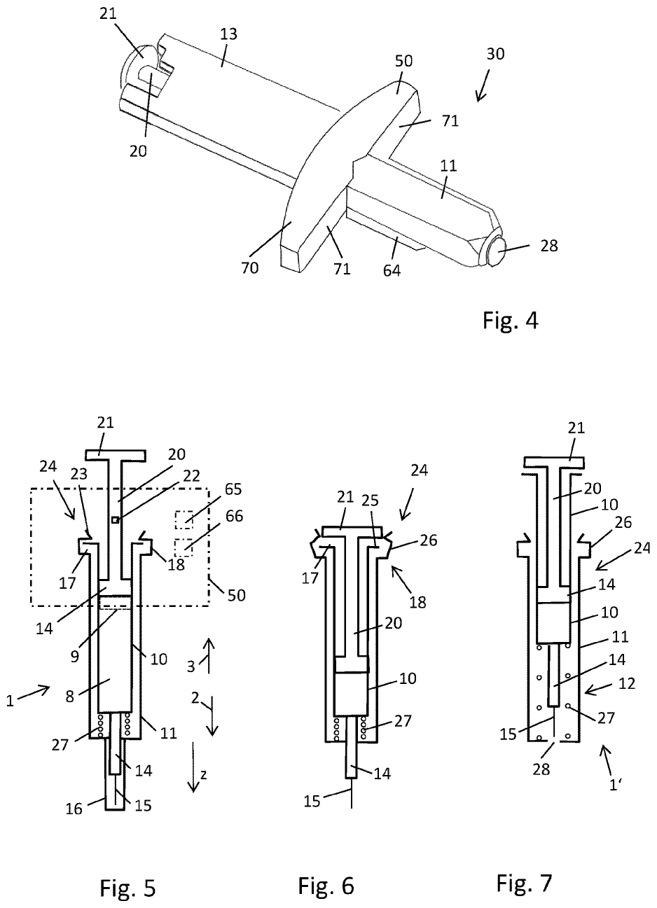

Optionally, and as e.g. apparent from the shielded configuration as shown in FIG. 4 the barrel 10 of the syringe may be encapsulated or mounted in a syringe carrier 13, which syringe carrier 13 is operably engaged with the spring element 27. The syringe carrier 13 may provide a dedicated sliding function inside the housing 11 and may house the barrel 10 inside.

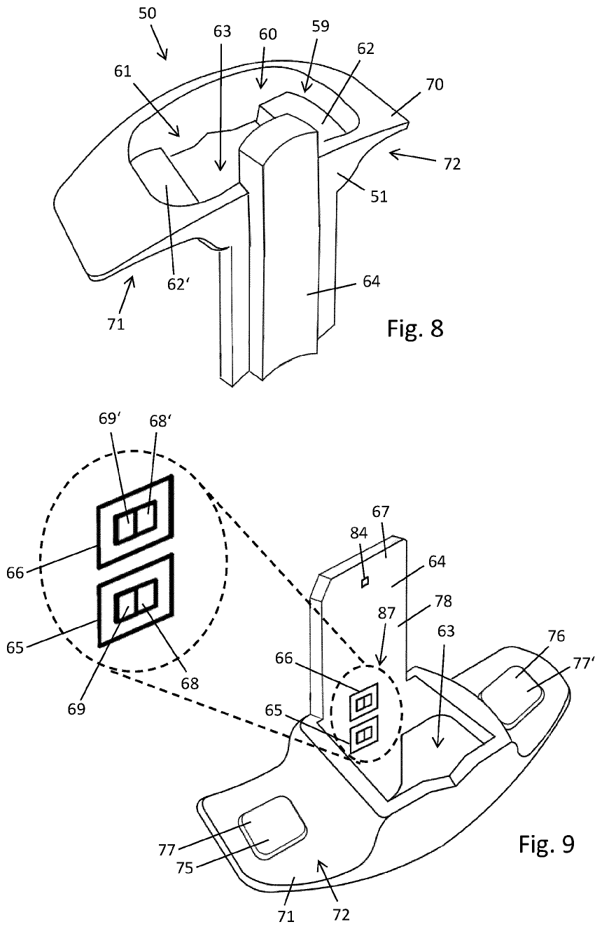

The auxiliary device 50 is schematically illustrated separately in FIG. 1 as well as in FIGS. 8, 9, 11 and 12. The auxiliary device 50 comprises a body 51 and comprises a fastening structure 60 by way of which the body 51 can be detachably connected to the injection device 1. Vice versa, by way of the fastening structure 60 the injection device 1 can be fastened or fixed to the body 51 of the auxiliary device 50. As shown in FIG. 8 the fastening structure 60 comprises a receptacle 61 with a lateral abutment 62 or abutment structure. The receptacle 61 and the abutment 62 are shaped and configured to receive the flange portion 18 with the distally facing abutment face 19 of the injection device 1 as illustrated in FIG. 1.

The receptacle 61 may comprise a through opening 63 to receive a portion, e.g. a distal portion of the injection device 1 there through as it is apparent from FIG. 2-4 or 10.

Inside the receptacle 63 there may be provided a mechanical coding 59 matching with a mechanical counter coding 29 as provided at or near the flange portion 18 of the housing 11 of the injection device 1. Here, the mechanical counter coding 29 may comprise the radially outwardly protruding flange portions 18′, 18″, which may differ in size or shape so as to fit into the receptacle 61 in only one or in a limited number of selected predefined orientation. For this, the abutment 62 or respective abutment shoulder on the inside of the receptacle 61 may be complementary shaped to the distally facing abutment faces 19 of the flange portion 18. In this way, there can be provided a unequivocal mounting or fastening configuration for fastening or attaching the injection device 1 to the auxiliary device 50.

The body 51 of the auxiliary device 50 further comprises a handle portion 70. The handle portion 70 comprises a distally facing gripping surface 71. The gripping surface 71 is provided on a gripping flange 72 protruding radially outwardly from the elongated structure of the body 51 or barrel 10 when attached to the elongated injection device 1. Optionally and as illustrated e.g. in FIG. 8 the body 51 comprises an elongated appendix 64, which when attached to the injection device 1 aligns with the elongated housing 11 or barrel 10 of the injection device 1.

As shown in FIGS. 2 and 3, the appendix 64 extends along a sidewall of the housing 11. As e.g. illustrated shown in FIG. 9, the appendix 64 may be of a slat-like profile and extends longitudinally from a sidewall 78 of the receptacle 61. With the injection device 1 assembled inside the receptacle 61 or through opening 63 at least a portion of the sidewall of the housing 11 of the injection device 1 extends along an inside 78 of the appendix 64, which inside faces towards the interior of the receptacle 61 or through opening 63. When correctly attached or mounted in the receptacle 61, the distally facing abutment faces 19 of the flange portion 18 of the injection device 1 are in longitudinal abutment with the proximally facing abutments 62, 62′ as illustrated in FIG. 8. In this way, the injection device 1 is fixed to the auxiliary device 50 at least with regard to the longitudinal direction (z) and hence with regard to the distal direction 2.

For injecting a dose of the medicament 8 a user has to exert a distally directed pressure onto the plunger flange 21 as illustrated in FIG. 10. Here, the user typically uses two fingers, e.g. the index finger 6 and the middle finger 7 of the same hand 4 to provide a counterforce against the pressure as applied by the thumb 5.

The handle portion 70 of the auxiliary device 50 mimics or replicates the typical flange portion 17, 18 of the injection device 1. For ease of use the size of the handle portion 70 and hence the size of the gripping flange 72 may be substantially larger compared to the respective size of the flange portions 17, 18. On a distally facing gripping surface 71 of the handle portion 70 and hence of the gripping flange 72 there is provided a first actuating element 75 and a second actuating element 70. The actuating elements 75, 76 may be implemented as electrical switches 77, 77′. The electrical switches 77, 77′ may be operably connected to an actuation unit 88 of an electronic circuit 80 of the auxiliary device 50.

On the inside sidewall 78 of the body 51 there are provided first and second sensors 65, 66. There may be further provided a transceiver 84, e.g. implemented as a second transceiver as described above. There may be further provided a further sensor, namely a third sensor 37.

When the injection device 1 and the auxiliary device 50 are suitably mounted or mutually connected the first and the second sensors 65, 66 may align with a dedicated portion or with a dedicated component of the injection device 1. By way of example and as e.g. illustrated in FIG. 5, the first sensor 65 may longitudinally align with a marking 22 provided on the plunger 20. The second sensor 66 may be aligned with the flange portion 17 and/or with a respective syringe carrier 13 of the safety syringe. First and second sensors 65, 66 may be implemented as optical sensors.

With an exemplary scenario of use the first sensor 65 may be operable to detect a movement of the plunger 20 relative to the barrel 10 or relative to the housing 11. The first sensor 65 may be operable to generate a respective first sensor signal being indicative that the marking 22 has left its initial position. Generation of the respective first sensor signal by the first sensor 65 is hence indicative of the start of an injection procedure.

The second sensor 66 may be operable to detect the position, presence or movement of the barrel 10 or of a respective syringe carrier 13 as the barrel 10 is subject to a movement relative to the housing 11. The housing 11 is and remains fixed to the body 51 of the auxiliary device during the entire lifetime of the injection device 1. When reaching a configuration as illustrated in FIG. 7 a respective movement of a characteristic or dedicated portion of the barrel 10 and/or of the syringe carrier 13 can be detected by the second sensor 66. Accordingly, the electronic circuit 80 equipped with first and second sensors 65, 66 is operable to detect at least two different configurations or operational states of the injection device.

With some examples, the second sensor 66 may be also implemented to detect an end position of the barrel 10 or of the syringe carrier 13, which is reached when the injection procedure has completed as e.g. illustrated in FIG. 7.

With some examples at least one of the first sensor 65 and the second sensor 66 is implemented as an optical sensor. The respective sensors 65, 66 may each comprise an optical detector 69, 69′ as shown in the enlarged section of FIG. 9. Concurrently with a respective optical detector the respective sensors 65, 66 may be equipped with an own or with a separate light source 68, 68′. Typically, the light sources 68, 68′ are implemented as light emitting diodes (LED.

The first optical sensor 65 may be equipped with a first light source 68 and the second optical sensor 66 may be equipped with a second light source 68′. An operating wavelength of the light sources 69, 69′ or a wavelength or spectrum of light generated by the light sources 68, 68′ may significantly distinguish. The same may apply to the sensitivity or susceptibility of the optical detectors 69, 69′ of the respective optical sensors 65, 66. With one example, the light source 68 and the optical detector 69 of the first optical sensor 65 operate at a first wavelength or in a first spectral range of electromagnetic radiation which is non-overlapping with a second wavelength or spectral range of the light source 68′ and the corresponding optical detector 69′ of the second optical sensor 66. In this way, cross talk between the light source 68 of the first optical sensor 68 with the detector 69′ of the second optical sensor 66 can be effectively avoided; and vice versa.

With some examples the light sources 68, 68′ of the individual sensors 65, 66 are aligned with a dedicated portion or component of the injection device 1 when the injection device 1 is attached or assembled to the auxiliary device 50. A dedicated portion or component of the injection device 1, such as the barrel 10, the housing 11, the shield 12 for the syringe carrier 13 may be at least partially provided or manufactured from a translucent or at least partially transparent material. A suitable alignment of the light source 68, 68′ with any of these components may provide an illumination effect of the respective device component. Moreover, and when first and second optical sensors 65, 66 operate at different non-overlapping spectral ranges there can be provided various illumination effects that may assist a user in operating the auxiliary device and/or the injection device.

With some examples the first sensor 65 is longitudinally aligned with a plunger 20 or with a marking 22 provided on the plunger 20 when the injection device 1 is in a first configuration or operational state, as e.g. illustrated in FIG. 5. Here, the injection device 1 is in a configuration or condition prior to dose injection. In this configuration the light source 68 of the first sensor 65 may be aligned with the plunger 20 in such a way, so as to illuminate the plunger 20. With other examples and/or with other configurations the light source 68 of the first sensor 65 may be aligned with the barrel 10 or with the housing 11, wherein the barrel 10 and/or the housing 11 may be at least partially transparent such that these components might be illuminated by the light source 68.

Concurrently, the light generated by the light source 68 and propagating e.g. towards the plunger 20 and/or towards the barrel 10 or housing 11 may be reflected at any of these components and may be captured by the optical detector 69. Insofar, the sensor 65 with the light source 68 and the light detector 69 may provide a twofold function. On the one hand the combination of the light source 68 with the optical detector 69 may allow to detect the presence or position of a suitably light reflecting component of the injection device 1, such as the plunger 20, the barrel 10 or any other movable component, such as the stopper 9. On the other hand the light source 68 may be also used to illuminate a dedicated portion or a dedicated component of the injection device 1. The light sources 68, 68′ of the first sensor 65 and the second sensor 66 may be operable to emit electromagnetic radiation of different wavelength or of a distinguishable electromagnetic spectrum.

This way and when the first and the second optical sensors 65, 66 should be operated simultaneously, a degree of cross talk can be minimized. Hence, the optical detector 69 of the first optical sensor 65 may be substantially insensitive to radiation generated by the light source 68′ of the second optical sensor 66. Vice versa, the optical detector 69 of the first optical sensor 65 may be substantially insensitive to radiation generated by the light source 68′ of the second sensor 66.

With some examples the light sources 68, 68′ may be monochromatic. With other examples the light sources 68, 68′ may be implemented as a rather broadband light sources. They may comprise a multicolor light source, that may be selectively operable to emit electromagnetic radiation of different and non-overlapping spectral ranges. With some examples the light sources 68, 68′ may be operable to generate electromagnetic radiation in the visible range, e.g. in any or several of the following colors: blue, green, yellow, orange and/or red.

Furthermore and since the optical sensors 65, 66 with their light sources 68, 68′ may be controlled by the processor 82 of the electronic circuit 80 it is conceivable that one of the first light sources 68, 68′ is operable to illuminate a component or portion of the injection device 1 in a first color at a first point in time and to eliminate the same or another component or portion of the injection device 1 in a different color in a second point of time. With some examples and when the injection device 1 is in a pre-use condition or configuration as e.g. illustrated in FIG. 2 or 5 at least one of the light sources 68, 68′ is operable to illuminate a portion or a component of the injection device 1 e.g. in a first color. In a different configuration as e.g. shown in FIG. 7 and when for instance the second sensor 66 detects a respective second configuration or operational status of the injection device the respective light sources 68, 68′ may be operable to eliminate the same or any other portion or component of the injection device 1 in a different color, e.g. red.

In this way and when illuminating selected or dedicated portions or components of the injection device 1, the user can be intuitively assisted in using the auxiliary device 50, the injection device 1 and/or the injection system 30.

In FIG. 10 a typical scenario of use of the injection system 30 is schematically illustrated. In FIG. 10 the injection device as illustrated in a configuration according to FIG. 7, namely after use of the injection device 1 and after retraction of the injection needle 15 into the housing 11 of the injection device 1.

Here and before reaching the illustrated configuration the user holds the injection system 30 in one hand, whereby the index finger 6 and the middle finger 7 of the hand 4 of the user are in abutment with the diametrically oppositely located portions of the gripping flange 72 of the body 51 of the auxiliary device 50. As shown in FIG. 10 the tips of the fingers 6, 7 are in direct abutment with first and second actuating elements 75, 76 as shown in FIG. 9. The gripping surface 71 faces distally, i.e. towards the distal end of the housing 11. With a thumb 5 the user may exert a distally directed pressure onto the plunger flange 21 of the plunger 20.

After completion of the injection procedure as illustrated in FIG. 10, the barrel 10 or a syringe carrier 13 protrudes proximally from the proximal end of the body 51 since the spring 27 located between the barrel 10 and housing 11 urges the barrel 10 in proximal direction 3. The first and the second actuating element 75, 76 may be both implemented as a mechanical or electromechanical switch 77. Both switches 77, 77′ may belong to an actuation unit 88 as schematically illustrated in the block diagram of the electronic circuit 80 of FIG. 8.

The two switches 77, 77′ and hence the first and the second actuating elements 75, 76 may form or constitute a kind of a logic AND gate. Accordingly, and by way of the first and second actuating element 75, 76 the electronic circuit 80 can be switched from an inactive mode into an operating mode. For this, the actuating elements 75, 76 have to be manually activated simultaneously. Actuation of only one of the actuating elements 75, 76 will be insufficient to switch the electronic circuit 80 from an inactive mode into the operating mode.

When implemented as switches 77, 77′, the switches may be arranged in series in the actuation unit 88. The actuation unit may be directly or indirectly connected with the processor 82 of the electronic circuit 80 and to a power source 81, e.g. in form of a battery, which may be rechargeable. The actuation unit and/or the two actuating elements 75, 76 may be connected to the processor 82 only or exclusively via the actuation unit 88. This means that the processor 82 or the processing unit of the electronic circuit 80 can be switched off, decoupled or disconnected from electrical power as soon one of the actuating elements 75, 76 is released by a user.

In this way, the likelihood of an and unintended and hence inadvertent activation of the electronic circuit 80, namely when a user only inadvertently touches or depresses one of the actuating element 75, 76 can be substantially reduced.

The electronic circuit 80 as shown in FIG. 8 further comprises a detection unit 87. The detection unit 87 comprises at least one of the sensors 65, 66. Optionally, the detection unit 87 also comprises the third sensor 67, which is operable to detect the presence or position of the protective cap 16. The detection unit 87 is operably connected to the processor 82.

The processor 82 is further connected to a memory 85. The memory is a digital memory. It may be implemented as a volatile or as a non-volatile memory. The electronic circuit 80 further comprises a clock 86. The clock 86 is operable to generate clock signals being indicative of a date and/or of a time. In a typical scenario of use and when the electronic circuit is in the operating mode and when the detection unit 87 detects at least one of a position or movement of a dedicated device component being indicative of e.g. the beginning or completion of a dose dispensing procedure a clock signal being indicative of a point of time may be used as a timestamp together with a respective indication that a dose of a particular size has been administered. This dosing information, typically together with the timestamp may be stored in the memory 85.

The processor 82 may be further configured to process different signals as provided by the detection unit 87. With some examples the first detector 65 may be operable to provide a first sensor signal while the second sensor 66 is operable to provide a second sensor signal. The first and the second sensor signals may be processed by the processor 82. In turn, the processor 82 may be operable to decide if the determined sensor signal are consistent with each other and belong to one and the same dose injection event. Only if a respective consistency check will be asserted a respective dose injection data may be generated and/or stored in the memory 85.

The electronic circuit 80 further comprises a first transceiver 83 and a second transceiver 84. The transceivers 83, 84 may be connected to the processor 82 and may provide communication with other electronic devices outside the electronic circuit 80. With some examples the first transceiver 83 is operable to transmit or to exchange data with an external electronic device as illustrated in FIG. 11. The external electronic device 100 may be implemented as a smart phone. The external electronic device 100 comprises a housing 101 and may be implemented as a portable electronic device. The external electronic device 100 further comprises a device processor 102 and a display 104. Optionally, the external electronic device 100 comprises a speaker and/or or a haptic user interface. In addition, the external electronic device 100 comprises a communication unit 106 operable to communicate with the first transceiver 83. With some examples the first transceiver 83 comprises a local range transceiver having a transmission range of several meters or tens of meters.

The further transceiver 84 is a near field transceiver and is operable to exchange data with a complementary near field electronic identifier 33 as provided on the injection device 1. Typically, and when the injection device 1 is suitably arranged or fixed to the auxiliary device 50 the electronic identifier 33 may be in close vicinity to the second transceiver 84, at least within the transmission range of the second transceiver 84.

The electronic identifier 33 may comprise electronic information or data, such as a name of the medicament, a name of the pharmaceutical substance of the medicament, a concentration of the medicament, a batch number, a LOT number, a manufacturing date, a manufacturing site or manufacturing location, an expiry date and/or a temperature, at which the medicament should be stored or has been stored. The respective information or data is readable by the second transceiver 84. The respective data can be further processed by the processor 82 and can be transmitted via the first transceiver 83 to the external electronic device 100. In this way, the auxiliary device 50 may act as a range extender for reading of an electronic identifier 33 of the injection device 1. Readout of the electronic identifier 33, which may be implemented as a near field communication tag (NFC tag), may be provided by the second transceiver 84 of the auxiliary device 50. Information or data thus obtained can be further processed by the processor 82 and can be transmitted to the external electronic device 100 via the first transceiver 83.

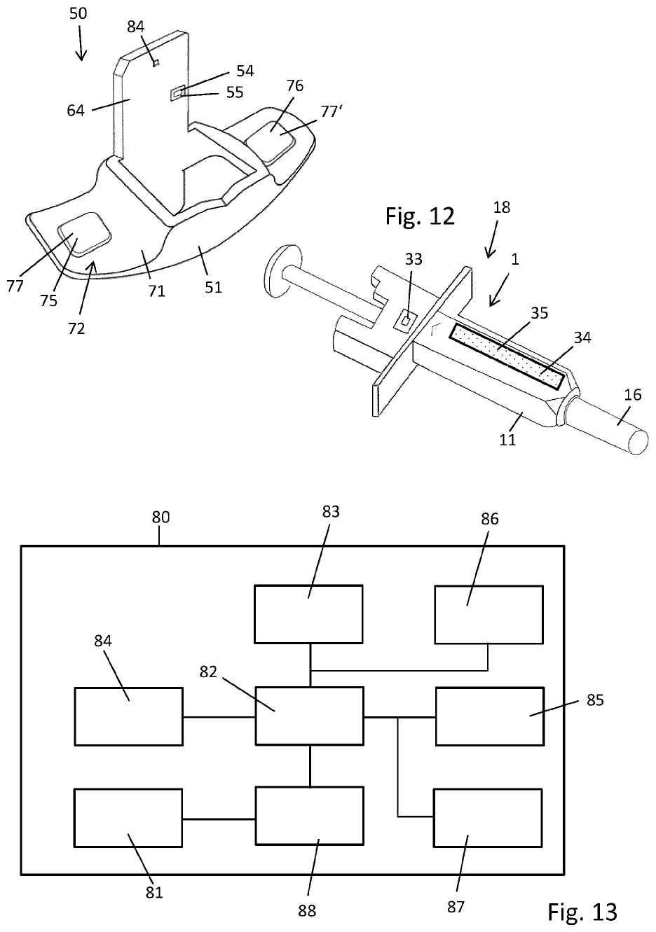

With the further example as illustrated in FIG. 12 the injection device 1 is equipped with a visual identifier 34, which may be provided on an outside surface of the housing. When the injection device 1 is correctly assembled with the auxiliary device 50 at least a portion of the visual identifier 34 may coincide or overlap with an indicator 54 as provided on the body 51 of the auxiliary device 50. The indicator 54 may be provided on an inside 78 of the body 51 facing towards an outside of the injection device 1, when the injection device 1 is correctly assembled or attached to the auxiliary device 50.

As illustrated in FIG. 12 the indicator 54 may be provided as a light source 55 operable to illuminate the visual identifier 34. The visual identifier 34 may comprise a longitudinally extending light guide 35 or a light-guiding structure 35 of any other shape or geometry. The visual identifier 34 may comprise a frosted surface of frosted structure and may be operable to reflect light emitted by the indicator 54 or light source 55 in a diffuse manner.

The indicator 54 or light source 55 may be connected to the processor 82 and may be operable through the processor 82. Here, and depending on the detected configurations of operational states of the injection device 1, the processor 82 may be configured to generate visible optical signals, e.g. light signals of varying duration, varying frequency or color to provide a respective visual effect along the visual identifier 34 or light guide 35.

A temporally varying illumination with regards to illumination time or duration as well as with regard to a variation in intensity or color may further assist a user in using the injection device 1, the auxiliary device 50 and/or the injection system 30 comprising the injection device 1 and the auxiliary device 50.

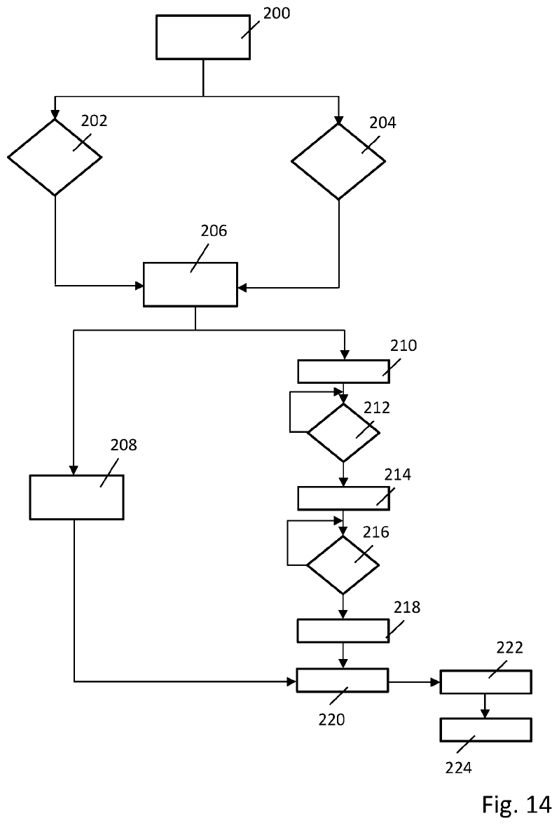

The flowchart of FIG. 14 shows a possible scenario of using the injection system 30, which comprises the injection device 1 assembled or attached to the auxiliary device 50 as described above. Here, and in a first step 200 the injection device 1 is attached and/or fixed to the auxiliary device 50.

In step 202 actuation of the first actuating element 75 is monitored or detected. In step 204 actuation of the second actuating element 76 is monitored or detected. Only if both activating elements 75, 76 are activated by a user, e.g. when respective switches 77, 77′ are pressed by respective fingers 6, 7 of a hand 4 of the user the electronic circuit 80 of the auxiliary device 50 is switched from the inactive mode into the operating mode in step 206.

Once the electronic circuit 80 has switched into the operating mode the method may continue with step 208. Here, the electronic identifier 33 as provided on or in the body 51 of the auxiliary device 50 may be read out by the electronic circuit 80. Specifically and as described above, the second transceiver 84 may read electronic data provided by the electronic identifier 33 of the injection device 1, which may be implemented as an electronic tag, such as a near field tag.

In step 210 the first sensor 65 is activated and is configured to detect a movement or position of a first component of the injection device 1. In a subsequent step 212 the first sensor 65 and/or the processor 82 connected to the first sensor 65 conducts a check or an assertion if the first component of the injection device 1 has moved or is currently subject to an expected movement. If and as long as no movement is detectable in step 212 the method returns to step 210. The loop of steps 210, 212 continues as long as the first sensor 65 detects an expected movement or a dedicated position of the first component of the injection device 1.

In the subsequent step 214 a respective timestamp or clock signal is generated by the clock 86 and the respective clock signal or timestamp may be stored in the memory 85 of the electronic circuit 80.

The method may then continue with step 216, in which the second sensor 66 is active or in which the first sensor 65 is activated again and becomes operable to detect a movement or position of the first component or of a second component of the injection device 1. Also here, and as long as an expected position or movement is not detected by the respective sensor 65, 66, step 216 is repeated multiple times as long as the expected movement or position of the first or second component of the injection device 1 is detected in step 216. If an expected movement or position of the first or second component of the injection device 1 is detected by any of the first and second sensors 65, 66, the method continues with step 218. Also here, and similar to step 214 a respective timestamp is generated by the clock 86 and the timestamp or clock signal is stored in the memory 85.

The timestamps or clock signals being indicative of e.g. the beginning and the completion of a dose injection procedure may be combined with medicament related injection device related information as obtained in step 208. In step 220 the data obtained from the electronic identifier 33 may be matched or combined with the timestamps as provided in steps 214 and 218. In step 222 the combination of the obtained data may be then stored in the local memory 85 of the electronic circuit 80. In a further subsequent and hence optional step 224 the data stored in the memory 85 may be finally transmitted to the external electronic device 100, e.g. for further data processing.

### REFERENCE NUMBERS

1 injection device
2 distal direction
3 proximal direction
4 hand
5 thumb
6 finger
7 finger
8 medicament
9 stopper
10 barrel
11 housing
12 shield
13 syringe carrier
14 outlet
15 needle
16 protective cap
17 flange portion
18 flange portion
19 abutment face
20 plunger
21 plunger flange
22 marking
23 beveled edge
24 interlock
25 latch element
26 counter latch element
27 spring element
28 through opening
29 mechanical counter coding
30 injection system
33 electronic identifier
34 visual identifier
35 light guide
50 auxiliary device
51 body
54 indicator
55 light source
59 mechanical coding
60 fastening structure
61 receptacle
62 abutment
63 through opening
64 appendix
65 sensor
66 sensor
67 sensor
68 light source
69 optical detector
70 handle portion
71 gripping surface
72 gripping flange
75 actuating element
76 actuating element
77 switch
78 sidewall
80 electronic circuit
81 power source
82 processor
83 transceiver
84 transceiver
85 memory
86 clock
87 detection unit
88 actuation unit
100 external electronic device
101 housing
102 device processor
104 display
106 communication unit

1-19. (canceled)

20. An auxiliary device for an injection device, the auxiliary device comprising:
a body comprising a fastening structure for detachably fastening to the injection device and comprising a handle portion for gripping by a user,
an electronic circuit, comprising a processor and a first transceiver, and switchable between an operating mode and an inactive mode,
a first actuating element provided on the handle portion, operably connected to the electronic circuit and operable to activate the electronic circuit when actuated by the user.

21. The auxiliary device according to claim 20, further comprising a second actuating element provided on the handle portion, operably connected to the electronic circuit and operable to activate the electronic circuit when actuated by the user.

22. The auxiliary device according to claim 21, wherein the electronic circuit is switchable from the inactive mode into the operating mode only by actuating the first actuating element and the second actuating element.

23. The auxiliary device according to claim 21, wherein the electronic circuit remains in the inactive mode if only one of the first and second actuating elements are actuated by the user.

24. The auxiliary device according to claim 21, wherein switching the electronic circuit from the inactive mode into the operating mode requires an actuation of both, the first actuating element and the second actuating element, in an at least temporally overlapping time interval.

25. The auxiliary device according to claim 21, wherein the electronic circuit is switchable from the operating mode into the inactive mode by releasing one or both of the first actuating element and the second actuating element.

26. The auxiliary device according to claim 21, wherein the first actuating element comprises a first switch, wherein the second actuating element comprises a second switch and wherein the first switch and the second switch are connected in series.

27. The auxiliary device according to claim 20, wherein the handle portion comprises a distally facing gripping surface and wherein the first actuating element is arranged on the distally facing gripping surface.

28. The auxiliary device according to claim 20, further comprising a first sensor operable to detect at least one of a position and a movement of a plunger or stopper of the injection device relative to one of a barrel, a housing and a shield of the injection device when the auxiliary device is fastened to the injection device.

29. The auxiliary device according to claim 28, further comprising a second sensor operable to detect at least one of a position and a movement of a plunger or stopper of the injection device relative to one of a barrel, a housing and a shield of the injection device when the auxiliary device is fastened to the injection device.

30. The auxiliary device according to claim 28, wherein the first sensor comprises an optical sensor.

31. The auxiliary device according to claim 30, wherein the first sensor comprises a first light source or wherein the first sensor is coupled with a first light source.

32. The auxiliary device according to claim 31, wherein the first light source is aligned with a first dedicated portion of the injection device when the injection device is correctly attached to the body and wherein the first light source is operable to illuminate the dedicated portion of the injection device.

33. The auxiliary device according to claim 28, further comprising a third sensor operable to detect at least one of a presence, a position and a movement of a protective cap of the injection device when the auxiliary device is fastened to the injection device.

34. The auxiliary device according to claim 20, further comprising a second transceiver operable to read an electronic identifier provided on or inside the injection device

35. The auxiliary device according to claim 34, wherein the second transceiver comprises a near field transceiver and wherein the first transceiver comprises a local range transceiver and wherein a transmission range of the first transceiver is larger than a transmission range of the second transceiver.

36. The device according to claim 20, wherein the inactive mode of the electronic circuit is a sleep mode, in which energy consumption of the processor is reduced compared to situations, in which the processor and/or the electronic circuit is in the operating mode.

37. An injection system comprising:
an injection device with a barrel filled with a medicament and sealed towards a proximal direction by a stopper movable in a distal direction relative to the barrel to expel the medicament through an outlet of the barrel,
an auxiliary device comprising
a body comprising a fastening structure for detachably fastening to the injection device and comprising a handle portion for gripping by a user,
an electronic circuit, comprising a processor and a first transceiver, and switchable between an operating mode and an inactive mode,
a first actuating element provided on the handle portion, operably connected to the electronic circuit and operable to activate the electronic circuit when actuated by the user.

38. A method of monitoring use or preparation of an injection device or injection system for injecting a medicament, the method comprising:
providing an injection device operable to inject the medicament and an auxiliary device
a body comprising a fastening structure for detachably fastening to the injection device and comprising a handle portion for gripping by a user,
an electronic circuit, comprising a processor and a first transceiver, and switchable between an operating mode and an inactive mode,
a first actuating element provided on the handle portion, operably connected to the electronic circuit and operable to activate the electronic circuit when actuated by the user, and
activating the electronic circuit of the auxiliary device by actuating the first actuating element.
